(19) United States
(12) Patent Application Publication
(10) Pub. No.: US 2005/0113766 A1
(43) Pub. Date: May 26, 2005

(54) Primer bulb for contrast media delivery system

(76) Inventors: Jim Mottola, South Jordan, UT (US); Steve W. Carlstrom, Salt Lake City, UT (US); Kirk L. Foote, Salt Lake City, UT (US); Penny B. Goldman, Murray, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/724,427

(22) Filed: Nov. 26, 2003

(51) Int. Cl. A61M 5/00

(52) U.S. Cl. 604/248; 600/431; 604/403

(57) ABSTRACT

The present invention relates to a primer bulb for use with a contrast media delivery system. The contrast media delivery system is configured to facilitate the intravenous delivery of contrast media from a contrast media reservoir to a patient. The primer bulb is configured to facilitate the flow of contrast media from the contrast media reservoir. The primer bulb is compressed by the user to create a head of pressure above the contrast media in the contrast media reservoir. The head of pressure overcomes the surface tension of the contrast media while drawing contrast media into the length of tubing positioned between the primer bulb and the contrast media reservoir.

### BACKGROUND OF THE INVENTION

1. The Field of the Invention

The present invention relates to contrast media delivery systems. In more particular, the present invention relates to a primer bulb adapted to facilitate the delivery of contrast media to a patient from a contrast media reservoir.

2. The Relevant Technology

The ability to monitor the flow of fluids within a patient's body systems is an important diagnostic tool in modem medicine. By being able to monitor the flow of fluids, a doctor can ascertain the nature and location of abnormalities within a patient. For example, by tracking the flow of blood through a patient's circulatory system potential problems such as arterial blockages or hemorrhaging can be detected and treated.

Contrast media provides a way of monitoring the flow of fluids in a patient's body. Contrast media facilitates the monitoring of movement of fluids utilizing x-ray, magnetic resonance imaging (MRI), and other contrast media monitoring systems. To monitor the flow of fluids, contrast media is introduced into the body system to be observed such as the circulatory system or digestive system. Once the contrast media is introduced into the system the contrast media mixes, and begins to circulate, with fluids of the body system. The properties of the contrast media allow special imaging equipment such as x-ray or magnetic resonance imaging to detect the presence of the contrast media as it moves through the system to be studied. For example, contrast media having a radioactive isotope can be introduced into the circulatory system of a patient. Progression of the contrast media through the circulatory system can be observed to detect abnormalities such as blockages, hemorrhaging, or the like. A variety of types of contrast media can be utilized in such diagnostic settings. Additional, the type of contrast media can be tailored to the body system or specific procedure to be performed on the patient.

One problem associated with the delivery of contrast media to a patient relates to the high viscosity of many types of contrast media. Typically the contrast media is stored in a reservoir such as a bottle or other container. A delivery system delivers the contrast media from the reservoir to the patient. A spike connected to the length of tubing is inserted into the contrast media reservoir. However, the high viscosity of the contrast media results in a surface tension that can prevent the flow of contrast media into the spike and length of tubing.

The effect of the surface tension of the contrast media can be exacerbated where the contrast reservoir includes a small amount of contrast media. The smaller amount of volume of contrast media typically results in a smaller head height of the fluid. The smaller head height exerts a smaller amount of pressure on the contrast media at the point of flow into the spike and length of tubing. In some instances practitioners shake, tap, or otherwise manipulate the contrast media reservoir in the attempt to facilitate the flow of contrast media into the spike and the length of tubing. This can result in turbulence and the formation of microscopic air bubbles in the contrast media reservoir. Smaller head heights of contrast media can result where most of the contrast media in the reservoir has already been utilized. Additionally, there is a trend in the market toward utilizing smaller contrast media reservoirs. This use of smaller reservoirs can result in smaller head heights of contrast media even in unused contrast media reservoirs.

A variety of systems have been developed to overcome the surface tension of contrast media. In one system a drip chamber is connected directly to the spike inserted into the contrast media reservoir. The drip chamber can be compressible allowing the user to squeeze the drip chamber so as to draw contrast media from the contrast media reservoir into the drip chamber. One difficult associated with drip chambers is that drip chambers are not always configured to fill completely with contrast media. The contrast media drips into the partially filled drip chamber, which can cause turbulence in the collection of contrast media in the drip chamber. The turbulence in the collection of contrast media in the drip chamber can result in the formation of microscopic bubbles. Such microscopic bubbles can be undesirable, particularly where the contrast media is delivered intravenously into the circulatory system of the patient.

Another drawback of utilizing a drip chamber is that the relatively large volume of contrast media in the drip chamber is often discarded when the drip chamber is disposed of after the procedure has been performed. Discarding of the contrast media can be costly due to the expense of even small quantities of contrast media. This is due to the unique properties of contrast media that allow its detection by imaging equipment. Additionally, the rarity of materials having the properties required to make contrast media and/or the specialized techniques required to manufacture the contrast media can make the loss of contrast media expensive. Another problem with utilizing drip chambers is that the design of the drip chambers can interfere with the creation of sufficient pressure to maintain flow of contrast media during procedures that require the practitioner to periodically stop and restart the flow of contrast media into the patient.

### BRIEF SUMMARY OF THE INVENTION

The present invention relates to a primer bulb for use with a contrast media delivery system. The contrast media delivery system is configured to facilitate the intravenous delivery of contrast media from a contrast media reservoir to a patient. The primer bulb is configured to facilitate the flow of contrast media from the contrast media reservoir. The primer bulb is compressed by the user to create a head of pressure above the contrast media in the contrast media reservoir. The head of pressure overcomes the surface tension of the contrast media while drawing contrast media into the length of tubing positioned between the primer bulb and the contrast media reservoir. The contrast media in the tubing can create a siphoning effect facilitating the flow of contrast media from the contrast media reservoir into the proximal portions of the contrast media delivery system.

The present invention also relates to a method of using a primer bulb to facilitate the delivery of contrast media from the contrast media reservoir to a patient. In the method, the primer bulb is connected to a contrast media reservoir. The primer bulb and the contrast media reservoir are then isolated from air pressurization of the external environment. The primer bulb is compressed creating a head of pressure in the contrast media reservoir to facilitate the flow of contrast media. The primer bulb and the contrast media reservoir are then opened to the air pressurization of the external environment to facilitate the flow of contrast media from the reservoir to the proximal portions of the contrast media delivery system.

According to one aspect of the present invention, the primer bulb is utilized with a vented spike apparatus. The vented spike apparatus is inserted into the contrast media reservoir to access contrast media from the contrast media reservoir. The vented spike apparatus can include a door or valve mechanism, which can be closed to isolate the primer bulb and contrast media reservoir from the air pressurization of the external environment. A length of tubing is positioned between the vented spike apparatus and the primer bulb such that contrast media is drawn into the length of tubing subsequent to compression of the primer bulb. The door or valve mechanism can be opened to facilitate the flow of air into the contrast media reservoir to allow the free flow of contrast media from the contrast media reservoir into the delivery system.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a perspective view of a contrast media delivery system illustrating a primer bulb for use with the contrast media delivery system.

FIG. 2A is a cross-sectional view of the primer bulb for use with a contrast media delivery system.

FIG. 2B is a cross-sectional view of a vented spike apparatus utilized in connection with the primer bulb of the contrast media delivery system.

FIG. 3A is a cross-sectional perspective view of a contrast media delivery system illustrating components of the contrast media delivery system.

FIG. 3B is a cross-sectional perspective view of the contrast media delivery system illustrating a compressed primer bulb.

FIG. 3C is a cross-sectional perspective view of the contrast media delivery system in which the primer bulb has been released and the flow of contrast media from the contrast media reservoir has been effectuated.

FIG. 3D is a perspective cross-sectional view of the contrast media delivery system illustrating the primer bulb filled with contrast media.

FIG. 4 is a perspective view of a contrast media delivery system having a primer bulb of an alternative configuration.

### DETAILED DESCRIPTION OF THE PREFERRED EMBODIMENTS

The present invention relates to a primer bulb for use with a contrast media delivery system. The primer bulb is configured to facilitate the flow of contrast media from the contrast media reservoir. The primer bulb is compressed by the user to create a head of pressure above the contrast media in the contrast media reservoir. The head of pressure overcomes the surface tension of the contrast media while drawing contrast media into a length of tubing positioned between the primer bulb and the contrast media reservoir. The contrast media in the tubing can create a siphoning effect facilitating the flow of contrast media from the contrast media reservoir into the proximal portions of the contrast media delivery system.

The present invention also relates to a method of using a primer bulb to facilitate the delivery of contrast media from the contrast media reservoir to a patient. In the method, the primer bulb is connected to a contrast media reservoir. The primer bulb and the contrast media reservoir are then isolated from air pressurization of the external environment. The primer bulb is compressed creating a head of pressure in the contrast media reservoir to facilitate the flow of contrast media. The primer bulb and the contrast media reservoir are then open to the air pressurization of the external environment to allow the flow of contrast media from the reservoir to the proximal portions of the contrast media delivery system.

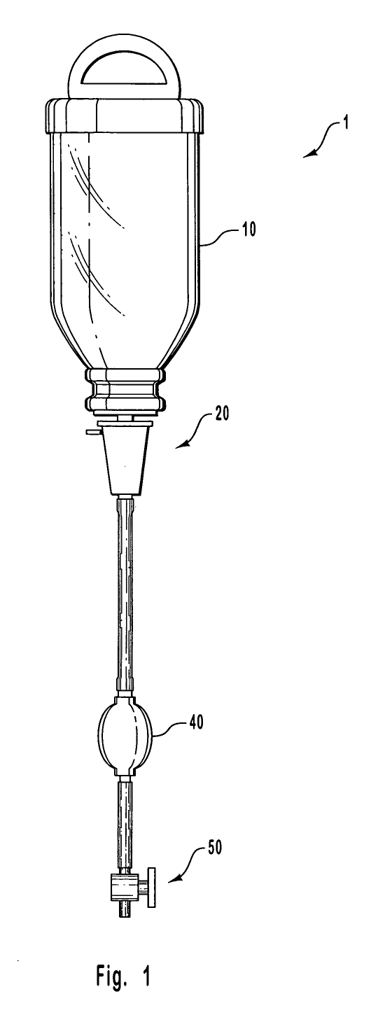

FIG. 1 illustrates a contrast media delivery system 1 having a primer bulb 40 according to one aspect of the present invention. The contrast media delivery system 1 facilitates delivery of contrast media to a patient in a safe and efficient manner. A variety of types and configurations of contrast media delivery systems can be utilized for the delivery of contrast media. Additionally, such contrast media delivery systems can be configured to facilitate the delivery of contrast media to different body systems (i.e. circulatory, digestive) and to different locations within those systems. In the illustrated embodiment, contrast media delivery system 1, includes a contrast media reservoir 10, a vented spike apparatus 20, a primer bulb 40, and a stop cock 50.

Contrast media reservoir 10 provides a mechanism for holding contrast media to be delivered to a patient. In the illustrated embodiment, contrast media reservoir 10 comprises a glass or rigid plastic bottle. Contrast media reservoir 10 is one example of a source of contrast media. As will be appreciated by those skilled in the art, a variety of types and configurations of sources of contrast media can be utilized without departing from the scope and spirit of the present invention. For example, in one embodiment, the contrast media reservoir comprises a pliable bag filled with contrast media. In another embodiment the source of contrast media is another mechanism or receptacle for holding an amount of contrast media.

Vented spike apparatus 20 is connected to contrast media reservoir 10. Vented spike apparatus 20 provides a mechanism for accessing contrast media in contrast media reservoir 10. Vented spike apparatus 20 also provides a mechanism for allowing air to enter contrast media reservoir 10 to facilitate the flow of contrast media into proximal portions of the contrast media delivery system 1. In the illustrated embodiment, vented spike apparatus 20 includes a spike member 30. Spike member 30 is inserted into contrast media reservoir 10 to access contrast media in contrast media reservoir 10.

Vented spike apparatus 20 is connected to primer bulb 40 utilizing a length of tubing. Primer bulb 40 is compressible to create a head of pressure to facilitate the flow of contrast media from contrast media reservoir 10. In the illustrated embodiment, primer bulb 40 comprises a pliable bulb and is positioned between contrast media reservoir 10 and the patient.

Stop cock 50 is positioned proximally to primer bulb 40. Stop cock 50 provides a mechanism for isolating primer bulb 40 and contrast media reservoir 10 from the air pressurization of the external environment. In the illustrated embodiment, stock cop 50 comprises a three-way stop cock having a first position, second position, and a third position. The first position comprises an off position in which primer bulb 40 and contrast media reservoir 10 are isolated from air pressurization of the external environment. The second position comprises an in-line position allowing the flow of contrast media from portions of the contrast media delivery system 1 distal to stop cock 50 to portions of the contrast media delivery system 1 proximal to stop cock 50. The third position comprises a bleed-off position in which contrast media is allowed to flow from portions of contrast media delivery system 1 distal to stop cock 50 to the external environment. The third position can be utilized to remove air bubbles, impurities, or unused portions of contrast media from the portions of contrast media delivery system distal to stop cock 50.

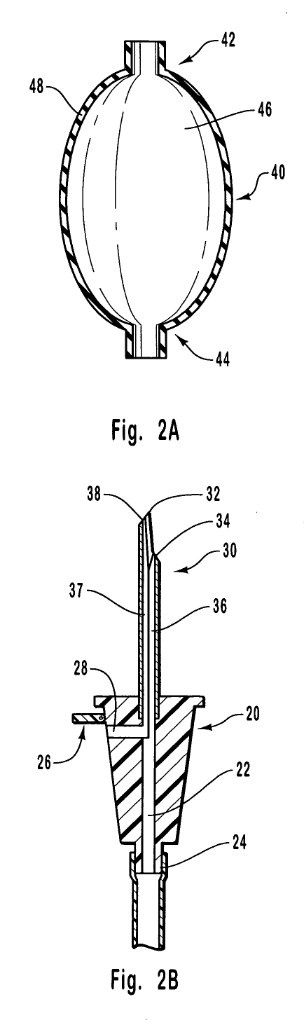

FIG. 2A is a cross-sectional view of primer bulb 40 according to one aspect of the present invention. In the illustrated embodiment, primer bulb 40 includes a top end 42, a bottom end 44, an inner cavity 46, and an outer wall 48. Top end 42 is contiguous with a portion of tubing positioned between primer bulb 40 and contrast media reservoir 10. Top end 42 is the portion of primer bulb 40 in which the contrast media first enters primer bulb 40. Bottom end 44 of primer bulb 40 is the portion of primer bulb 40 positioned adjacent a portion of tubing connected between primer bulb 40 and stop cock 50. As contrast media enters primer bulb 40 from top end 42 it moves to bottom end 44. Once the contrast media reaches bottom end 44 it begins to fill primer bulb 40 from bottom end 44 to top end 42. While contrast media fills primer bulb 40 it can exit primer bulb 40 by means of tubing adjacent bottom end 44. As will be appreciated by those skilled in the art, the speed with which contrast media fills primer bulb 40 from top end 42 to bottom end 44 is at least in part dependent upon the viscosity of the contrast media being utilized and can vary without departing from the scope and spirit of the present invention.

Inner cavity 46 is positioned between top end 42 and bottom end 44. Inner cavity 46 is defined by outer wall 48. The volume of inner cavity 46 can be selected to provide a predetermined amount of pressurization in contrast media reservoir 10 when primer bulb 40 is compressed. Outer wall 48 of primer bulb 40 is comprised of a pliable material such as, but not limited to, polyurethane, rubber, or the like.

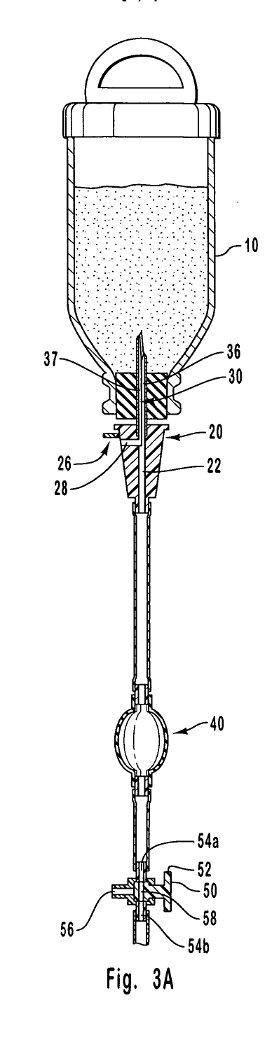

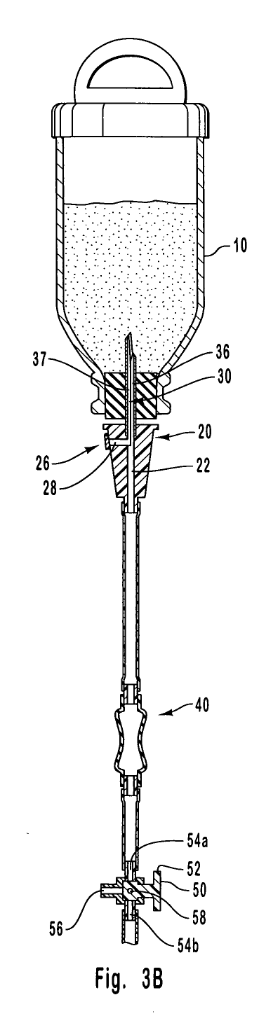

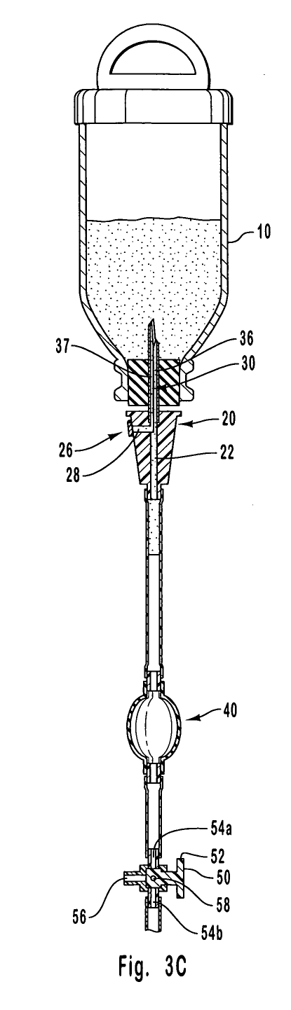

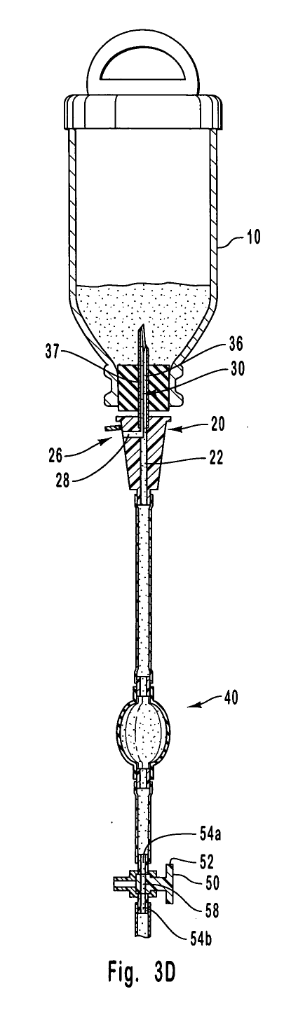

As will be appreciated by those skilled in the art, a variety of types and configurations of primer bulbs can be utilized without departing from the scope and spirit of the present invention. For example, in one embodiment the primer bulb is positioned in fluid coupling with the other components of the contrast media delivery system in other than an in-line configuration. In another embodiment, primer bulb 40 has a rectangular rather than an elliptical shape. In anther embodiment, the primer bulb includes a substantially rigid housing having a depressible mechanism or other component so as to change the volume of the primer bulb inner cavity. The functionality of the primer bulb will be discussed in greater detail with reference to FIG. 3A-3D.

FIG. 2B is a cross-sectional view of vented spike apparatus 20 of FIG. 1 illustrating the manner in which vented spike apparatus 20 operates in connection with contrast media delivery system 1. Vented spike apparatus 20 provides a mechanism for accessing contrast media from the contrast media reservoir 10 while also allowing venting of air into contrast media reservoir 10 to facilitate the flow of contrast media from contrast media reservoir 10 to proximal components of contrast media delivery system 1. In the illustrated embodiment, vented spike apparatus 20 comprises an inner lumen 22, a tube engagement member 24, a venting door 26, a side venting opening 28, and a spike member 30.

Spike member 30 provides a mechanism for accessing contrast media from contrast media reservoir 10. An insertion tip 32 is inserted into contrast media reservoir 10 to provide direct fluid coupling with contrast media in contrast media reservoir 10. Once insertion tip 32 of spike member 30 is inserted into contrast media reservoir 10, the flow of contrast media from contrast media reservoir 10 can be facilitated by the primer bulb. When contrast media in contrast media reservoir 10 begins to flow the contrast media moves from drainage bore 34 to inner lumen 36. From inner lumen 36 contrast media moves to inner lumen 22 and then to the tubing coupled to tube engagement member 24 of vented spike apparatus 20.

As contrast media moves from the contrast media reservoir 10 to the proximal portions of the contrast media delivery system through vented spike apparatus 20, the decreasing amount of fluid in the contrast media reservoir 10 can result in a vacuum affect in contrast media reservoir 10. This can impede the flow of contrast media, particularly when the contrast media reservoir is a rigid container such as a glass bottle or other rigid plastic contrast media storage device. A venting lumen 37 is linked to side venting opening 28 to allow for the flow of air from the external environment into the contrast media reservoir. The venting provided by venting lumen 37 and side venting opening 28 allows for the pressurization in the contrast media reservoir 10 to equalize. By allowing the pressurization in the contrast media reservoir 10 to equalize, flow of contrast media from contrast media reservoir 10 can continue uninhibited by pressurization differentials. Thus, to overcome the vacuum affect a user can open venting door 26 allowing air to move from venting door 26 through side venting opening 28 and venting lumen 37 to venting bore 38. This can equalize the pressurization of the air in contrast media reservoir 10 with that of the air pressurization of the external environment. By positioning venting bore 38 higher than drainage bore 34, contrast media can flow from contrast media reservoir 24 through drainage bore 34 while also allowing air to flow from side venting opening 28 through venting lumen 37 to venting bore 38.

As will be appreciated by those skilled in the art, a variety of types and configurations of vented spike apparatuses can be utilized without departing from the scope and spirit of the present invention. For example, in one configuration, the vented spike apparatus comprises an apparatus having a one-way venting valve. In another embodiment, the spike apparatus is not vented but merely allows drainage of contrast media from the contrast media reservoir 10.

FIG. 3A is a cross-sectional view of contrast media delivery system 1 illustrating the configuration of contrast media delivery system 1. In the illustrated embodiment, the surface tension of the contrast media in contrast media reservoir 10 prevents the flow of contrast media into the proximal portions of contrast media delivery system 1. The contrast media in contrast media reservoir 10 is in direct fluid contact with spike member 30 of vented spike apparatus 20. The venting door 26 of vented spike apparatus 20 is opened allowing the direct exchange of air pressurization of the external environment with contrast media reservoir 10 and primer bulb 40.

A selector handle 52 of stop cock 50 is positioned such that an upper main channel 54a of stop cock 50 is in fluid communication with a lower main channel 54b of stop cock 50. In the illustrated configuration the positioning of selector handle 54 is such that valve member 58 isolates main channel 54 from side channel 56. This allows the portions of contrast media delivery system 1 positioned proximally to stop cock 50 to be in fluid communication with primer bulb 40 and contrast media reservoir 10. Positioning of selector handle 52 allows a direct path of fluid communication from contrast media reservoir 10 to the proximal portions of contrast media delivery system 1.

The path of fluid communication flows from contrast media reservoir 10 through inner lumen 36 of spike member 30 to inner lumen 22 of vented spike apparatus 20. From the inner lumen of vented spike apparatus 20 the fluid communication continues to the tubing positioned between vented spike apparatus 20 and primer bulb 40 and then continues to the inner cavity 46 of primer bulb 40. From the inner cavity 46 of primer bulb 40 it continues to the tubing positioned between primer bulb 40 and stop cock 50 and then continues to main channel 54 of stop cock 50. While a path of fluid communication is open from contrast media reservoir 10 to the main channel of stop cock 50, the surface tension of the contrast media in contrast media reservoir 10 prevents the flow of contrast media from contrast media reservoir 10.

FIG. 3B illustrates contrast media delivery system 1 in which primer bulb 40 can facilitate the flow of contrast media from contrast media reservoir 10. In the illustrated embodiment, venting door 26 of vented spike apparatus 20 is closed to isolate contrast media reservoir 10 and primer bulb 40 from air pressurization of the external environment. Additionally, selector handle 52 of stop cock 50 has been turned to the first position (off position) such that valve member 58 blocks fluid communication between upper main channel 54a, lower main channel 54b, and side channel 56. In this configuration contrast media reservoir 10 and primer bulb 40 are isolated from the external environment and from the portions of contrast media delivery system 1 positioned proximal to stop cock 50.

With selector handle 52 of stop cock 50 in the first position and venting door 26 of vented spike apparatus 20 being closed, primer bulb 40 is compressed. When primer bulb 40 is compressed, the volume of the inner cavity 46 of primer bulb 40 is substantially reduced. By reducing the volume of the inner cavity 46, the pressurization of the portions of the contrast media delivery system 1 in fluid communication with primer bulb 40 are substantially increased. In particular, the pressurization of the air above the contrast media in contrast media reservoir 10 is increased.

When primer bulb 40 is released from its compressed configuration inner cavity 46 of primer bulb 40 returns to its pre-compression volume decreasing the pressurization in primer bulb 40, vented spike apparatus 20, and the tubing position therebetween. While the air pressurization in primer bulb 40, vented spike apparatus 20 and the tubing position therebetween is lessened, the pressurization of the air in contrast media reservoir 10 remains at a higher level due to the fluid barrier provided by the contrast media in contrast media reservoir 10. The differential in air pressurization between the air in contrast media reservoir 10 and the portions of contrast media delivery system 1 proximal to contrast media reservoir 10 results in a head of pressure which exerts a downward force on the contrast media in contrast media reservoir 10. The head of pressure in the contrast media reservoir 10 is designed to overcome the surface tension of the contrast media in contrast media reservoir 10. This allows the flow of contrast media into the portions of contrast media delivery system 1 positioned proximal to contrast media reservoir 10.

FIG. 3C illustrates contrast media delivery system 1 in which primer bulb 40 is returned to its non-compressed configuration. In the illustrated embodiment, contrast media from contrast media reservoir 10 has moved into portions of contrast media delivery system 1 positioned proximal to contrast media reservoir 10. Movement of contrast media from contrast media reservoir 10 into portions of contrast media delivery system 1 positioned proximal to contrast media reservoir 10 results from the head of pressure created by compression of primer bulb 40. The head of pressure created by compression of primer bulb 40 overcomes the surface tension of contrast media in contrast media reservoir 10. By overcoming the surface tension of contrast media in contrast media reservoir 10, contrast media can flow from contrast media reservoir 10 into vented spike apparatus 20 and from vented spike apparatus into a portion of the tubing positioned between vented spike apparatus 20 and primer bulb 40.

As the head of pressure exerts downward force on contrast media in contrast media reservoir 10, movement of contrast media from contrast media reservoir 10 increases the volume of air in contrast media reservoir 10 while decreasing the volume of air in the portions of contrast media delivery system positioned proximately to contrast media reservoir 10. The flow of contrast media from contrast media reservoir 10 continues until the change in the volume of air in contrast media reservoir 10 and air in the portions of contrast media delivery system 1 positioned proximal to contrast media reservoir 10 equalize and the head of pressure in contrast media reservoir 10 substantially decreases.

Additional movement of contrast media from contrast media reservoir 10 to the portions of the contrast media delivery system positioned proximately to contrast media reservoir 10 can create an air pressure differential. The air pressure differential results as movement of contrast media begins to compress the air in primer bulb 40 while reducing the pressurization in contrast media reservoir 10. As the negative air pressure differential is created, the flow of contrast media is stopped and the formation of a partial vacuum pressure in contrast media reservoir 10 is formed. This is due to the fact that venting of pressurization into contrast media reservoir 10 is prevented by closure of venting door 26 of vented spike apparatus 20 and valve member 58 of stop cock 50.

FIG. 3D illustrates contrast media deliver system 1 in which contrast media is flowing from contrast media reservoir 10 to the portions of contrast media delivery system 1 positioned proximal to stop cock 50. In the illustrated embodiment, venting door 26 is opened such that contrast media reservoir 10 is positioned in fluid communication with the air pressurization of the external environment. Additionally, selector handle 52 is moved such that valve member 58 allows fluid communication of upper main channel 54a and lower main channel 54b. Opening of venting door 26 allows air pressurization from the external environment to flow through side venting opening 28 into contrast media reservoir 10. The mechanics of fluid transfer from side venting opening 28 to contrast media reservoir 10 are discussed in greater detail with reference to FIG. 2B. The flow of air pressurization from the external environment to contrast media reservoir 10 reduces the likelihood that the air pressurization in contrast media reservoir 10 will be reduced below the air pressurization of the external environment in a manner that might prevent the flow of contrast media from the contrast media reservoir 10 to the proximal portions of the contrast media delivery system 1.

The fluid coupling of upper main channel 54a with lower main channel 54b of stop cock 50 prevents the compression of air in the portions of contrast media delivery system 1 positioned proximal to stop cock 50. The presence of the contrast media in vented spike apparatus 20 and the tubing position between vented spike apparatus 20 and primer bulb 40 creates a siphoning effect maintaining the flow of contrast media from contrast media reservoir 10. With venting door 26 opened and valve member 58 positioned such that upper main channel 54a and lower main channel 54b are in fluid communication, any pressurization differential between contrast media reservoir 10 and primer bulb 40 that may otherwise stop the flow of contrast media from contrast media reservoir 10 is eliminated.

In the illustrated configuration, it can be seen that contrast media has flowed from contrast media reservoir 10 to the portions of contrast media delivery system 1 proximal to stop cock 50. Additionally, primer bulb 40 is completely filled with contrast media and the amount of contrast media in contrast media reservoir 10 has decreased. As will be appreciated by those skilled in the art, a variety of types and configurations of contrast media delivery systems and methods can be utilized with the primer bulb without departing from the scope and spirit of the present invention.

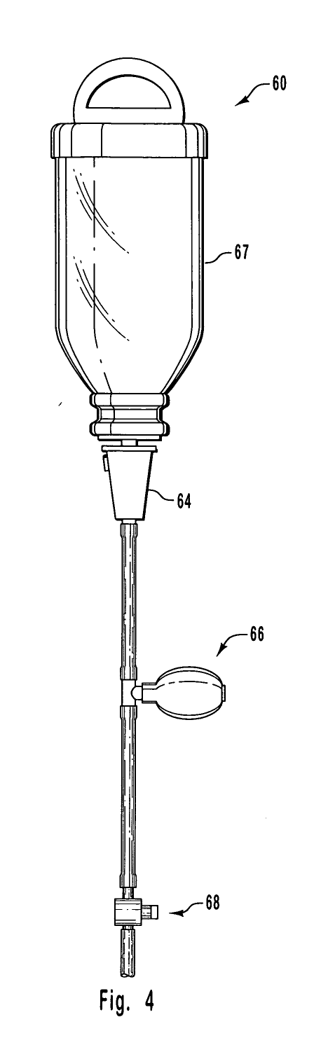

With reference now to FIG. 4, there is shown a contrast media delivery system according to another embodiment of the present invention. In the illustrated embodiment, contrast media delivery system 60 includes a contrast media reservoir 62, a vented spike apparatus 64, a primer bulb 66, and a stop cock 78. In the illustrated embodiment, primer bulb 66 is positioned between vented spike apparatus 64 and stop cock 68. Primer bulb 66 is positioned laterally to the portion of tubing positioned between vented spike apparatus 64 and stop cock 68. In this configuration, primer bulb 66 facilitates the flow of contrast media from contrast media reservoir 62. While primer bulb 66 facilitates the flow of contrast media from contrast media reservoir 62 to the portions of contrast media delivery system 1, primer bulb 66 may not completely fill with fluid as illustrated with respect to primer bulb 40 of FIGS. 1-3D.

A variety of types and configurations primer bulbs can be utilized without departing from the scope and spirit of the present invention. For example, in one embodiment the primer bulb comprises an enlarged portion of tubing positioned between the stop cock and the vented spike apparatus. In another embodiment, the primer bulb is utilized which can create a pressure differential between the air pressurization and the contrast media reservoir and the portions of the contrast media delivery system positioned proximally to the contrast media reservoir 10.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A contrast media delivery system configured to facilitate the intravenous delivery of contrast media from a contrast media source to a patient, the contrast media delivery system comprising:
a spike for accessing contrast media from the contrast media source;
a length of tubing linked to the spike;
a primer bulb in fluid coupling to the length of tubing, the primer bulb adapted to create a head of pressure in at least a portion of the length of tubing to facilitate flow of contrast media into the length of tubing.

2. The contrast media delivery system of claim 1, wherein the primer bulb is positioned in an in-line configuration of the length of tubing.

3. The contrast media delivery system of claim 1, wherein the primer bulb is positioned in an other than in-line configuration with the length of tubing.

4. The contrast media delivery system of claim 1, wherein the primer bulb is compressible.

5. The contrast media delivery system of claim 4, wherein compressing the primer bulb overcomes the surface tension of the contrast media in the contrast media delivery reservoir.

6. The contrast media delivery system of claim 5, wherein a single compression of the primer bulb overcomes the surface tension of the contrast media in the contrast media delivery reservoir.

7. The contrast media delivery system of claim 6, wherein more than one compression of the primer bulb overcomes the surface tension of the contrast media in the contrast media delivery reservoir.

8. The contrast media delivery system of claim 4, wherein compression of the primer bulb draws contrast media into the length of tubing.

9. The contrast media delivery system of claim 4, wherein the length of tubing and the primer bulb are isolated from the external environment before compression of the primer bulb.

10. The contrast media delivery system of claim 4, wherein compression of the primer bulb creates a head of pressure in the contrast media reservoir.

11. A contrast media delivery system configured to facilitate the intravenous delivery of contrast media from a contrast media reservoir to a patient, the contrast media delivery system comprising:
a contrast media reservoir;
a spike for accessing contrast media in the reservoir;
a length of tubing linked to the spike;
a primer bulb fluid coupled to the length of tubing, the primer bulb adapted to create a head of pressure to facilitate flow of contrast media into the length of tubing;
a valve mechanism providing a fluid tight seal to isolate the reservoir, length of tubing, and primer bulb from the inflow of air from the external environment.

12. The contrast media delivery system of claim 11, wherein the valve mechanism comprises a venting door.

13. The contrast media delivery system of claim 12, wherein the venting door is opened to allow the flow of contrast media.

14. The contrast media delivery system of claim 11, wherein the valve mechanism comprises a one-way valve.

15. The contrast media delivery system of claim 11, wherein the valve mechanism is linked to the spike.

16. The contrast media delivery system of claim 11, wherein the valve mechanism is integrally coupled to the spike.

17. The contrast media delivery system of claim 11, wherein the valve mechanism facilitates the flow of contrast media from the contrast media reservoir.

18. The contrast media delivery system of claim 17, wherein the valve mechanism facilitates the flow of air into the contrast media reservoir to allow the flow of contrast media from the contrast media reservoir.

19. The contrast media delivery system of claim 11, further comprising a stop cock.

20. The contrast media delivery system of claim 19, wherein the stop cock comprises a three-way stop cock.

21. The contrast media delivery system of claim 19, wherein the stop cock is positioned below the primer bulb.

22. A contrast media delivery system configured to facilitate the intravenous delivery of contrast media from a contrast media reservoir to a patient, the contrast media delivery system comprising:
a contrast media reservoir;
a spike for accessing contrast media in the reservoir;
a length of tubing linked to the spike;
a primer bulb connected in-line with the length of tubing, wherein the primer bulb is compressible to create a head of pressure above the contrast media in the contrast media reservoir to facilitate the flow of contrast media into the length of tubing;
a valve mechanism providing a fluid tight seal to isolate the reservoir, length of tubing, and primer bulb from the inflow of air from the external environment.

23. The contrast media delivery system of claim 22, wherein spike comprises a vented spike.

24. The contrast media delivery system of claim 23, wherein the vented spike apparatus allows air to enter the contrast media reservoir.

25. The contrast media delivery system of claim 24, wherein the vented spike facilitates the flow of contrast media into proximal portions of the contrast media delivery system.

26. A contrast media delivery system configured to facilitate the intravenous delivery of contrast media from a contrast media reservoir to a patient, the contrast media delivery system comprising;
a compressible primer bulb in fluid connection with the contrast media reservoir, wherein the primer bulb is configured to be completely filled with contrast media once the contrast media has reached the patient; and
a fluid delivery mechanism for delivering contrast media from the contrast media reservoir to the patient, wherein the primer bulb is positioned in-line with the fluid delivery mechanism.

27. A contrast media delivery system of claim 26, wherein the primer bulb includes an outer wall.

28. A contrast media delivery system of claim 27, wherein the outer wall defines an inner cavity.

29. A contrast media delivery system of claim 27, wherein the outer wall is comprised of a pliable material.

30. A contrast media delivery system of claim 29, wherein the outer wall is comprised of polyurethane.

31. A contrast media delivery system of claim 30, wherein the outer wall is comprised of rubber.

32. A contrast media delivery system of claim 28, wherein primer bulb comprises a rigid housing with a compressible mechanism configured to change the volume of primer bulb cavity.

33. A method for facilitating the delivery of contrast media from a contrast media reservoir to a patient, the method comprising the acts of:
connecting a primer bulb to a contrast media reservoir;
isolating the primer bulb and the contrast media reservoir from air pressurization from the external environment;
compressing the primer bulb to create a head of pressure in the contrast media reservoir to facilitate the flow of contrast media; and
opening the primer bulb and the contrast media reservoir to the air pressurization of the external embodiment.
